(19) United States
(12) Patent Application Publication
(10) Pub. No.: US 2012/0137574 A1
(43) Pub. Date: Jun. 7, 2012

(54) SYSTEMS AND METHODS FOR HARVESTING ALGAE

(75) Inventors: David Stephen, Davis, CA (US); Gaye Elizabeth Morgenthaler, Woodside, CA (US); Benjamin Chiau-pin Wu, San Ramon (CA)

(73) Assignee: LiveFuels, Inc., California (US)

(21) Appl. No.: 13/375,633

(22) PCT Filed: Jun. 15, 2010
(86) PCT No.: PCT/US10/38627

§ 371 Date: Feb. 23, 2012

Related U.S. Application Data
(60) Provisional application No. 61/187,571, filed on Jun. 16, 2009.

(51) Int. Cl. C10L 1/18; C12M 1/00; C07C 51/09

(52) U.S. Cl. 44/388; 554/8; 435/289.1

(57) ABSTRACT

Provided herein are systems and methods for producing biofuel from microalgae that use a population of zooplankton to harvest microalgae in a culture. The methods further comprise gathering the zooplankton, extracting lipids from the zooplankton, and processing the lipids to form biofuel. The systems provided herein comprise at least one enclosure comprising microalgae, means for making the microalgae available to a population of zooplankton, and means for gathering the zooplankton.

This application claims the benefit of U.S. Provisional Application No. 61/187,571, filed Jun. 16, 2009, which is incorporated by reference in its entirety.

### 1. INTRODUCTION

Provided herein are systems and methods for harvesting microalgae used in the production of useful lipids and biofuels.

### 2. BACKGROUND OF THE INVENTION

The large-scale culturing of algae presents a feasible option for producing useful lipids and biofuels, but there is a need to reduce the cost of operating an algae culture facility and producing the lipids and biofuels from algae. Provided herein are cost-effective and energy-efficient approaches for harvesting algae.

### 3. SUMMARY OF THE INVENTION

Provided herein are systems and methods directed to using zooplankton to harvest microalgae for the production of useful lipids and biofuels. The methods provided herein comprise feeding microalgae to zooplankton, gathering the zooplankton, extracting lipids from the zooplankton, and processing the lipids to produce useful lipids and biofuels. In certain embodiments, the methods provided herein further comprise feeding the zooplankton to zooplanktivorous fish, gathering these fish, extracting lipids from the fish, and processing fish lipids to produce useful lipids and biofuels. In some embodiments, zooplankton can be used in conjunction with phytoplanktivorous fish to harvest microalgae. The systems and methods provided herein are also useful for regulating the structure of a plankton community in a body of water.

### 4. DETAILED DESCRIPTION OF THE INVENTION

Use of phytoplanktivorous fish to harvest microalgae is more economical than harvesting mechanically and is efficient in terms of energy transfer, because no intermediate trophic level is involved. However, microalgae differ in linear dimensions by more than four orders of magnitude, and phytoplanktivorous fish do not have the ability to filter microalgae of smaller size ranges. Therefore, smaller microalgae that are not harvested by phytoplanktivorous fish represent a loss in biomass that could be used to produce useful lipids and biofuels. Those uneaten smaller size ranges of microalgal species also utilize nutrients and are likely to result in a significant loss of biomass. Under certain nutrient conditions and without grazing pressure, such microalgae can overgrow and may destabilize the higher trophic levels in the culture system.

The systems and methods provided herein are particularly useful in an open outdoor algae culturing facility, although they can be used with closed systems such as photobioreactors. The advantages in using zooplankton to harvest microalgae include (i) recovering more microalgal biomass, in the smaller size range of microalgae, from a culture when using only phytoplanktivorous fish, (ii) providing zooplankton as an additional food source for zooplanktivorous macro/megaplankton (zooplankton that feed on other zooplankton) or zooplanktivorous fish, (iii) providing the option of extracting lipids directly from zooplankton, and (iv) providing stability of plankton community structure.

The terms “microalgae” and “phytoplankton,” used interchangeably herein, refer to any microscopic algae, photoautotrophic or photoauxotrophic protozoa, photoautotrophic or photoauxotrophic prokaryotes, and cyanobacteria (commonly referred to as blue-green algae and formerly classified as Cyanophyceae). In certain embodiments, systems and methods provided herein can be practiced with a planktonic composition, without isolation of the phytoplankton, or removal of other non-algal planktonic organisms. In certain embodiments, the methods provided herein can be used with a composition comprising plankton, or a body of water comprising plankton.

In an algae culture system provided herein, autotrophic and heterotrophic plankton respond to nutrient supplies, and a trophic-level relationship or a food chain emerges. In certain embodiments, bacteria that thrive on dissolved organic matter (DOM) and autotrophic picoplankton and nanoplankton form the lowest level 1 of a trophic web. Ciliates and heterotrophic flagellates feed on organisms on level 1 and are in turn preyed on by zooplankton, such as copepods and cladocerans. Larger microplankton and mesoplankton are foods for copepods, cladocerans, and certain planktivorous fishes. Planktonic tunicates feed on bacteria, picoplankton, and nanoplankton, as well as heterotrophic nanoflagellates. Copepods, cladocerans, and tunicates are in turn food for adult zooplanktivorous fishes and for larval planktonic stages of fishes and crustaceans and mollusces (e.g., cephalopods).

In certain embodiments, methods provided herein comprise using one or more groups of zooplankton to harvest microalgae in culture or to regulate the structure of a microalgae community in a body of water. In some embodiments, the methods comprise monitoring the population diversity and density of zooplankton in the system or body of water, and introducing additional zooplankton species and/or adding more zooplankton to the system. In some embodiments, depending on the levels of nutrients and environmental conditions, certain groups of zooplankton may be preferred, and different groups of zooplankton may be introduced or removed from the system at various times. In certain embodiments, the methods provided herein comprise providing one or more population of zooplankton, each comprising one or more species, that feeds predominantly on microalgae of a certain size range. In some embodiments, the zooplankton can be used to harvest microalgae of a particular size range in a culture. In some embodiments, the dimensions of the harvesting zooplankton can be about twice, five times, ten times, 100 times, 10³ times or 10⁴ times greater than the dimensions of a dominant species of microalgae that is being harvested. In some embodiments, the zooplankton can be used to regulate the structure of a microalgae community in a body of water by selectively depleting or decreasing the density of microalgae of a particular kind or size range. Pelagic trophic relationships are governed mostly by the relative body sizes of feeding organisms (predator) and food organisms (prey). Plankton can be described in decadal size classes: picoplankton (<2 μm), nanoplankton (2 to 20 μm), microplankton (20 to 200 μm), mesoplankton (200 μm to 2 mm), macroplankton (2 mm to 2 cm), megaplankton (>2 cm). Generally, microalgae (also referred to as phytoplankton) are not represented in the megaplankton size range, zooplankton not in the picoplankton size range, and metazoans not in the pico- and nanoplankton size ranges. The term zooplankton, as used herein, encompasses meroplankton, such as larval stages of aquatic organisms, including ichthyoplankton. In certain embodiments, the zooplankton provided herein are pelagic mesozooplankton, which are about 200 μm to about 2 mm in dimensions, and include but are not limited to copepods, cladocerans, and tunicates. The systems and methods provided herein can be implemented with any freshwater, brackish water, marine, or hyperhaline species of microalgae and zooplankton. The zooplankton population provided herein can comprise one or more of the following taxonomic groups of aquatic organisms.

In certain embodiments, the population of zooplankton comprises cladocerans. Most cladocerans are freshwater filter feeders, e.g., Daphnia species, with a few genera that are limited to coastal waters. Generally, their metabolic and reproduction rates are high, and with ample food supply, mature cladocerans continue to grow in size. Daphnia have relatively low carbon:phosphorus (C:P) and nitrogen:phosphorus (N:P) stoichiometry than most freshwater zooplankton due to high RNA content. Cladocerans are known to select food only by size, e.g., Daphnia species from 1-30 μm; Penilia species from 2-100 μm. Fish that feed visually are predators of the larger cladocerans. Exemplary marine cladocerans include but are not limited to: Penilia, Podon, Evadne species. Exemplary freshwater cladocerans include but are not limited to Daphnia species (D. magna, D. obtuse, D. longispina, D. cucculata), Bosimina species (B. longirostris), and Sinobosmina species (S. leideri).

In certain embodiments, a population of zooplankton comprises copepods, preferably pelagic copepods, that occur both in marine and in freshwater environments, many of which belong to the suborder Calanoida. Generally, copepods are slower growing and have lower birth rates than claodocerans. Copepods prefer larger food particles than cladocerans, such as plankton in the 20 to 200 μm range. Copepods' food selection depends not only on size but also on biochemical basis. Copepods are known to avoid certain toxic algae. Exemplary marine copepods include but are not limited to: Calanus, Calanoides, Neocalanus, Pseudocalanus, Eucalanus, Rhincalanus, Euchirella, Euchaeta, Metridia, Acartia, and Centropages species. Exemplary freshwater copepods include, but are not limited to, Heterorocope and Eudiaptomus species.

In certain embodiments, the population of zooplankton comprises tunicates, preferably pelagic tunicates, that include salps, appendicularians, pyrosomas, and doliolids. Tunicates are filter feeders that as a group of organisms can filter the entire size range of microalgae. Generally, tunicates do not store much lipid reserves and can colonize oligotrophic waters. They do not develop diapause, but have very high growth rates. Tunicates are consumed by fish and fish larvae. Due to their high growth rate and the non-discriminating nature of their filtering ability, tunicates can affect the size structure of a plankton population. Many appendicularians, referred to as microphagous, feed on particles that are in the colloidal and bacterial size range, e.g., generally smaller than 5 μm which include nano- and picoplankton. Examples of microphagous appendicularians include, but are not limited to, Oikopleura species (O. dioica, O. vanhoeffeni). Also contemplated is the use of transgenic strains of O. dioica that possess desired characteristics, such as improved accumulation of lipids. Salps and doliolids feed on larger microplankton in the 20 μm to 200 μm range. Exemplary species of salps include, but are not limited to, Salpa species (S. fusiformis), Pegea species (P. confoderata), Thalia species (T. democratica). Exemplary species of pyrosomas include, but are not limited to, Pyrosoma atlanticum. Exemplary species of doliolids include, but are not limited to, Doliolum species (D. nationalis). It has been observed in a major bloom of toxic dinoflagellates in Tampa Bay, numerical abundance of copepods, Oithona and Acartia species declined, while the appendicularian Oikopleura dioica mimicked the pattern of dinoflagellate abundance (Badylak et al. 2008, J. Plankton Res., 30:449-465).

In certain embodiments, the population of zooplankton comprises meroplankton that are larval stages of aquatic organisms, which include, but are not limited to, fish, shellfish, crustaceans, mollusks, echinoderms, and polychaetes. In certain embodiments, the larval stages of crustaceans in the family Euphausiidae and Artemiidae are less preferred. In a preferred embodiment, the population of zooplankton comprises ichthyoplankton which are larval forms of fishes that are cultured commercially or that are cultured to harvest microalgae in the systems provided herein, including but not limited to, planktivorous, herbivorous, or omnivorous fishes of the order Clupeiformes, Siluriformes, Cypriniformes, Mugiliformes, and/or Perciformes. In certain embodiments, many fishes in the order Clupeiformes that are filter feeders are useful in the systems and methods provided herein and belong to the following families: Chirocentridae, Clupeidae (menhadens, shads, herrings, pilchards, sardines, hilsa), Denticipitidae, and Engraulidae (anchovies). Other groups of filter feeding fishes include, but are not limited to, shiners such as members of Luxilus, Cyprinella and Notropis genus. In certain embodiments, standard fish hatchery practices can be applied to produce ichthyoplankton provided herein.

In another embodiment, the population of zooplankton comprises trochophores and/or veligers of mollusks, such as gastropods and bivalves. Preferably, the zooplankton population comprises trochophores and/or veligers of bivalves that are cultured commercially, such as but not limited to members of Mytiloida (Mytilidae (sea mussels)); Ostreoida (Pectimidae, Ostreidae, Unionoida (freshwater mussels); Myoida, Pterioida (Pteriidae, pearl oysters), and Veneroida (Tridacnidae, Lucimidae). Among the gastropods are subclasses of organisms that produce pearl, e.g., Patellogastropoda, Vetigastropoda, Littprinoida, Tonnida, Muricoida, and Pulmonata.

In certain embodiments, one or more species of zooplankton can be used to harvest the microalgae. In one embodiment, the population of zooplankton comprises only one species. In another embodiment, the zooplankton population is mixed and thus comprises one or several major species of zooplankton. A major species is one that ranks high in the head count, e.g., the top one to five species with the highest head count relative to other species. In another embodiment, a mixed zooplankton population can be described and distinguished from other populations by the major species of zooplankton present. In some embodiments, the population of zooplankton can be further described by the percentages of the major and minor species, or the percentages of each of the major species. It is to be understood that in a body of water comprising a mixed zooplankton population having the same genus or species of zooplankton as another body of water may be different by virtue of the relative abundance of the various genus and/or species of zooplankton present.

In certain embodiments, the systems and methods provided herein can be used under various aquatic and environmental conditions, for example, in algae culture system that takes advantage of a water source with an abundance of nutrients. Nutrient-rich water due to upwelling contains high levels of nitrogen (N), phosphorous (P) and silicon (Si). Upwelling of nutrient-rich waters results in algal blooms, followed by decreases after depletion of nutrients. Upwelling occurs near continental margins, or it can be induced artificially. Upwelling can also be caused by seasonal vertical mixing in temperate and boreal seas. Diatoms are generally abundant in regions with upwelling. Copepods and cladocerans can be used advantageously to harvest population of microalgae that has an abundance of diatoms, as well as other heterotrophic zooplankton that feed on the microalgae. Diatoms are favored by high Si:N and Si:P ratios, while non-Si microalgae, e.g., dinoflagellates, are favored at low Si ratios. The changeover occurs at Si:N at 0.2:1 to 2:1 and Si:P at 3:1 to 30:1.

In certain embodiments, eutrophic coastal water is used. Eutrophic coastal water typically has a lower Si:N or Si:P ratio than upwelling, which is due to excess anthropogenic N and P. Under such eutrophic conditions, microalgae larger than diatoms, including colonial forms, are favored. Flagellates are also abundant and many such species are found in harmful algal blooms. Copepods and cladocerans can be used to harvest microalgae that grow well in eutrophic water but copepods are known to avoid ingesting toxic algae. Therefore, in certain embodiments, non-selective filter feeders, such as tunicates, can be used additionally for harvesting microalgae in such eutrophic water. In some embodiments, the abundance of detritus in eutrophic water, for example from cell lysis, provides food for a growing bacterial population. In certain embodiments, tunicates, with its ability to filter particles in the submicron range, are also capable of and used for harvesting bacteria and the heterotrophic nanoflagellates that feed on the bacteria.

In certain embodiments, oligotrophic water is used. In oligotrophic water in the open ocean, picoplankton and nanoplankton are dominant. In certain embodiments, for algae culture system established in oligotrophic water, tunicates, with appendicularians in particular, and certain cladocerans, are preferably used to harvest plankton in the size ranges of picoplankton and nanoplankton.

In certain embodiments, one or more species of microalgae can be harvested by the methods provided herein. In one embodiment, the composition comprising the microalgae is a monoculture, wherein only one species of algae is grown. In certain embodiments, open culturing systems are used, in which more than one algae species are present in the algal composition. In some embodiments, a mixed algal composition provided herein comprises one or several dominant species of microalgae. A dominant species is one that ranks high in the number of algal cells, e.g., the top one to five species with the highest number of cells relative to other species. Microalgae occur in unicellular, filamentous, or colonial forms. The number of algal cells can be estimated by counting the number of colonies or filaments. Microalgae inhabit all types of aquatic environment, including but not limited to freshwater, marine, and brackish environment, and all climatic regions, such as tropical, subtropical, temperate, and polar. Accordingly, in certain embodiments, the systems and methods provided herein can be practiced with algae and fishes in any of such aquatic environments and climatic regions. In certain embodiments, the systems and methods provided herein can be practiced in many parts of the world, including but not limited to the coasts, the contiguous zones, the territorial zones, and the exclusive economic zones of the United States. In some embodiments, a system provided herein can be established at the coasts of Gulf of Mexico, or in the waters of the Gulf of Mexico basin, Northeast Gulf of Mexico, South Florida Continental Shelf and Slope, Campeche Bank, Bay of Campeche, Western Gulf of Mexico, and/or Northwest Gulf of Mexico.

In certain embodiments, storage lipids provide zooplankton with energy for reproduction, periods of low food supply, obtaining food, escaping predation, and vertical migration. Triacylglycerols are more rapidly turned over for short term energy needs, while wax esters serve as long term energy deposits, especially in species found in high latitudes. Other lipids found in zooplankton include, but are not limited to, phospholipids and diacylglycerol ethers. Generally, algal fatty acids are incorporated unmodified into zooplankton storage lipids. Typically, there is a buildup of storage lipids in copepods, such as Calanus, Calanoides, Neocalanus species, during seasonal algal bloom following by decent into deeper water for diapause during non-upwelling periods. Accordingly, in certain embodiments, the methods provided herein comprise maintaining a sufficient supply of microalgae such that the zooplankton do not enter diapause. Alternatively, the methods comprise gathering the copepods before they enter diapause.

In certain embodiments, one or more population of zooplankton are used in parallel with fish to harvest microalgae. In certain embodiments, the methods comprise using both zooplankton and fish in the same body of water to harvest microalgae. In a preferred embodiment, the fish also feed on the zooplankton. In some embodiments, the zooplankton and the larvae of planktivorous fish are introduced into an algae culture. In some embodiments, the fish larvae or ichthyoplankton can feed on the microalgae and grow to maturation. In some embodiments, the mature planktivorous fish can feed on both the microalgae and the zooplankton. In another embodiment, a population of zooplankton is allowed to harvest microalgae, initially without the presence of planktivorous fish. In some embodiments, after a period of time sufficient for the zooplankton to feed on the microalgae, planktivorous fish are introduced to gather the microalgae and zooplankton. In certain embodiments, provided herein are methods for producing useful lipids or biofuels from microalgae, said method comprising: (i) providing one or more populations of zooplankton and one or more populations of planktivorous fishes; wherein zooplankton in at least one of said populations of zooplankton feed on microalgae, thereby harvesting the microalgae; (ii) gathering the planktivorous fishes in said one or more populations of planktivorous fishes that feed on (a) said one or more populations of zooplankton, and/or (b) said one or more populations of zooplankton and the microalgae; (iii) extracting lipids from (c) the zooplankton, and/or (d) the planktivorous fishes, that have been gathered; and (iv) processing the lipids to form useful lipids or biofuels.

In certain embodiments, more than one population of zooplankton are being used in series to harvest microalgae, preferably, starting with a first population of zooplankton that feed on microalgae of the smallest size class in the culture. The methods further comprise using a second population of zooplankton that feed on microalgae that are bigger than those in the smallest size class, and/or zooplankton in the first population. Successive populations of zooplankton can be used to harvest microalgae of increasing sizes and/or other smaller zooplankton. In a specific embodiment, a planktivorous fish is used to harvest zooplankton at the end of the series.

In certain embodiments, the fish that can be used to harvest zooplankton can be practiced with planktivorous or omnivorous fishes of the order Clupeiformes, Siluriformes, Cypriniformes, Mugiliformes, and/or Perciformes. In certain embodiment, at least one planktivorous species of fish in the order Clupeiformes are used. Non-limiting examples of useful fishes, including menhadens, shads, herrings, sardines, hilsas, anchovies, catfishes, carps, milkfishes, shiners, paddlefish, and/or minnows.

In another embodiment, zooplankton having dimensions that are greater than about 500 μm, about 1 mm, or about 2 mm can be gathered from the water directly without involving fish. In certain embodiments, the methods can use filtration, fractionation, and/or centrifugation.

In certain embodiments, any of the methods known in the art for culturing copepods (e.g. Calanus or Acartia species), cladocerans (e.g. Daphnia species, see Kilham et al. 1998, Hydrobiologia 377:147-159), and tunicates (e.g. salps, appendicularians and doliolids, see Raskoff et al. 2003, Biol. Bull. 204:68-80; Nishida, 2009, Develop. Growth Differ. 50:S239-256), can be used to maintain stocks of the organisms. See also, Plankton culture manual, F H Hoff, T W Snell-1989-fao.org; “Culture techniques for studies on the growth, development and reproduction of copepods and cladocerans under laboratory and in situ conditions: a review” by Vijberberg 1989, Freshwater Biology, 21:317.

In certain embodiments, depending on the latitude of the site of the system, microalgae obtained from tropical, subtropical, temperate, polar or other climatic regions are harvested by the methods provided herein. In certain embodiments, endemic or indigenous microalgal species are generally preferred over introduced species where an open culturing system is used. In certain embodiments, it is advantageous to use microalgae, zooplankton and/or fishes from a local aquatic trophic system in the methods provided herein.

As used herein, the term fish refers to a member or a group of the following classes: Actinopteryii (i.e., ray-finned fish) which includes the division Teleosteri (also known as the teleosts), Chondrichytes (e.g., cartilaginous fish), Myxini (e.g., hagfish), Cephalospidomorphi (e.g., lampreys), and Sarcopteryii (e.g., coelacanths). Transgenic fish and genetically improved fish can also be used in the harvesting methods provided herein. The term “genetically improved fish” refers herein to a fish that is genetically predisposed to having a higher growth rate and/or a lipid content that is higher than a wild type fish, when they are cultured under the same conditions. Such fishes can be obtained by traditional breeding techniques or by transgenic technology.

In certain embodiments, the systems provided herein comprise at least one enclosure comprising microalgae, means for feeding microalgae to a population of zooplankton, or both zooplankton and fishes, means for measuring zooplankton density or biomass, means for gathering the zooplankton, means for extracting lipids from the zooplankton, and means for converting the lipids into useful lipids or biofuels.

In certain embodiments, the systems provided herein comprise any means for feeding microalgae to a population of zooplankton that are known in the art.

In certain embodiments, the fishes and zooplankton can be gathered or harvested by any methods or means known in the art. In some embodiments, a fish and/or zooplankton gathering or capturing means is configured to separate fish and/or zooplankton based on a selected physical characteristic, such as density, weight, length, or size. The harvesting systems of the embodiments comprise means to gather or capture fish and/or zooplankton, which can be any mechanical, pneumatic, hydraulic, electrical, or a combination of mechanisms.

In certain embodiments, provided herein are products resulting from practicing the systems and methods, i.e., a composition comprising zooplankton lipids, said composition being prepared by extracting lipids from zooplankton that had been fed with cultured microalgae. In certain embodiments, useful lipids and biofuels are made by processing zooplankton lipids by methods known in the art.

In certain embodiments, provided herein are a biofuel feedstock or a biofuel comprising lipids, hydrocarbons, or both, derived from fish that harvested algae according to the methods provided herein. Lipids obtained by the systems and methods provided herein can be subdivided according to polarity: neutral lipids and polar lipids. The major neutral lipids are triglycerides, and free saturated and unsaturated fatty acids. The major polar lipids are acyl lipids, such as glycolipids and phospholipids. The hydrocarbons obtained by the systems and methods provided herein include, but are not limited to, isoprenoids, or pigments such as chrlorophyll, carotenoids (e.g., carotene, lycopene, lutein), astaxanthin, melanin, anthocyanins, porphyrins, tetraterpenoids, and betalains. A composition comprising lipids and hydrocarbons obtained by the systems and methods provided herein can be described and distinguished by the types and relative amounts of key fatty acids and/or hydrocarbons present in the composition.

Fatty acids are identified herein by a first number that indicates the number of carbon atoms, and a second number that is the number of double bonds, with the option of indicating the position of the first double bond or the double bonds in parenthesis. The carboxylic group is carbon atom 1 and the position of the double bond is specified by the lower numbered carbon atom. For example, linoleic acid can be identified by 18:2 (9, 12).

In certain embodiments, fatty acids produced by the cultured algae provided herein comprise one or more of the following: 12:0, 14:0, 14:1, 15:0, 16:0, 16:1, 16:2, 16:3, 16:4, 17:0, 18:0, 18:1, 18:2, 18:3, 18:4, 19:0, 20:0, 20:1, 20:2, 20:3, 20:4, 20:5, 22:0, 22:5, 22:6, and 28:1 and in particular, 18:1(9), 18:2(9, 12), 18:3(6, 9, 12), 18:3(9, 12, 15), 18:4(6, 9, 12, 15), 18:5(3, 6, 9, 12, 15), 20:3(8, 11, 14), 20:4(5, 8, 11, 14), 20:5(5, 8, 11, 14, 17), 20:5(4, 7, 10, 13, 16), 20:5(7, 10, 13, 16, 19), 22:5(7, 10, 13, 16, 19), 22:6(4, 7, 10, 13, 16, 19). Without limitation, it is expected that many of these fatty acids are present in the lipids extracted from the fishes that ingested the cultured algae. Algae produce mostly even-numbered straight chain saturated fatty acids (e.g., 12:0, 14:0, 16:0, 18:0, 20:0 and 22:0) with smaller amounts of odd-numbered acids (e.g., 13:0, 15:0, 17:0, 19:0, and 21:0), and some branched chain (iso- and anteiso-) fatty acids. A great variety of unsaturated or polyunsaturated fatty acids are produced by algae, mostly with C₁₂ to C₂₂ carbon chains and 1 to 6 double bonds, mainly in cis configurations.

The hydrocarbons present in algae are mostly straight chain alkanes and alkenes, and may include paraffins and the like having up to 36 carbon atoms. The hydrocarbons are identified by the same system of naming carbon atoms and double bonds as described above for fatty acids. Non-limiting examples of the hydrocarbons are 8:0, 9, 0, 10:0, 11:0, 12:0, 13:0, 14:0, 15:0, 15:1, 15:2, 17:0, 18:0, 19:0, 20:0, 21:0, 21:6, 23:0, 24:0, 27:0, 27:2(1, 18), 29:0, 29:2(1, 20), 31:2(1, 22), 34:1, and 36:0.

In certain embodiments, a great variety of unsaturated or polyunsaturated fatty acids are produced by fish mostly with C₁₂ to C₂₂ carbon chains and 1 to 6 double bonds, mainly in cis configurations (Stansby, M. E., “Fish oils,” The Avi Publishing Company, Westport, Conn., 1967). Fish oil comprises about 90% triglycerides, about 5-10% monoglycerides and diglycerides, and about 1-2% sterols, glyceryl ethers, hydrocarbons, and fatty alcohols. One of skill would understand that the amount and variety of lipids in fish oil varies from one fish species to another, and also with the season of the year, the algae diet, spawning state, and environmental conditions. Fatty acids produced by the fishes provided herein comprise, without limitation, one or more of the following: 12:0, 14:0, 14:1, 15:branched, 15:0, 16:0, 16:1, 16:2 n-7, 16:2 n-4, 16:3 n-4, 16:3 n-3, 16:4 n-4, 16:4 n-1, 17:branched, 17:0, 17:1, 18:branched, 18:0, 18:1, 18:2 n-9, 18:2 n-6, 18:2 n-4, 18:3 n-6, 18:3 n-6, 18:3 n-3, 18:4 n-3, 19:branched, 19:0, 19:1, 20:0, 20:1, 20:2 n-9, 20:2 n-6, 20:3 n-6, 20:3 n-3, 20:4 n-6, 20:4 n-3, 20:5 n-3, 21:0, 21:5 n-2, 22:0, 22:1 n-11, 22:2, 22:3 n-3, 22:4 n-3, 22:5 n-3, 22:6 n-3, 23:0, 24:0, 24:1 (where n is the first double bond counted from the methyl group). See, also Jean Guillaume, Sadisivam Kaushik, Pierre Bergot, and Robert Metailler, “Nutrition and Feeding of Fish and Crustaceans,” Springer-Praxis, UK, 2001).

In certain embodiments, provided herein are methods of making a liquid fuel which comprise processing lipids derived from fish that harvested algae. Products provided herein made by the processing of fish-derived biofuel feedstocks can be incorporated or used in a variety of liquid fuels including but not limited to, diesel, biodiesel, green diesel, kerosene, jet-fuel, gasoline, JP-1, JP-4, JP-5, JP-6, JP-7, JP-8, Jet Propellant Thermally Stable (JPTS), Fischer-Tropsch liquids, alcohol-based fuels including ethanol-containing transportation fuels, and other biomass-based liquid fuels including cellulosic biomass-based transportation fuels.

In certain embodiments, triacylglycerides in fish oil can be converted to fatty acid methyl esters (FAME or biodiesel), for example, by using a base-catalyzed transesterification process (for an overview see, e.g., K. Shaine Tyson, Joseph Bozell, Robert Wallace, Eugene Petersen, and Luc Moens, “Biomass Oil Analysis: Research Needs and Recommendations, NREL/TP-510-34796, June 2004). The triacylglycerides are reacted with methanol in the presence of NaOH at 60° C. for 2 hrs to generate a fatty acid methyl ester (biodiesel) and glycerol.

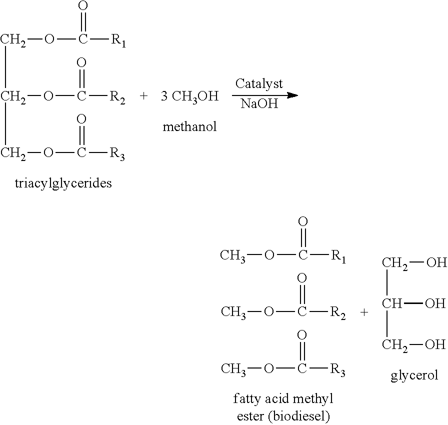

The biodiesel and glycerol co-products are immiscible and typically separated downstream through decanting or centrifugation, followed by washing and purification. Free fatty acids (FFAs) are a natural hydrolysis product of triglyceride and formed by the following reaction with triacylglycerides and water:

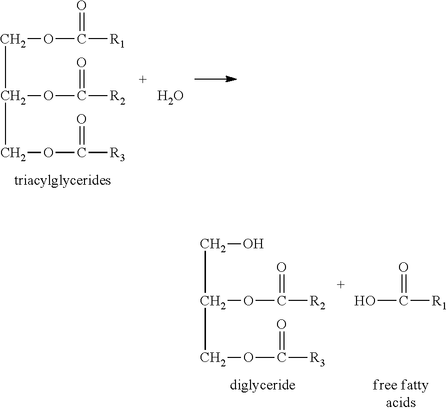

In some embodiments, this side reaction is undesirable because free fatty acids convert to soap in the transesterification reaction, which then emulsifies the co-products, glycerol and biodiesel, into a single phase. Separation of this emulsion becomes extremely difficult and time-consuming without additional cost-prohibitive purification steps.

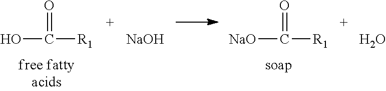

In certain embodiments, the methods provided herein can further comprise a step for quickly and substantially drying the fish oil by techniques known in the art to limit production of free fatty acids, preferably to less than 1%. In another embodiment, the methods provided herein can further comprise a step for converting or removing the free fatty acids by techniques known in the art.

In certain embodiments, triacylglycerides in fish oil can also be converted to fatty acid methyl esters (FAME or biodiesel) by acid-catalyzed transesterification, enzyme-catalyzed transesterification, or supercritical methanol transesterification. Supercritical methanol transesterification does not require a catalyst (Kusdiana, D. and Saka, S., “Effects of water on biodiesel fuel production by supercritical methanol treatment,” Bioresource Technology 91 (2004), 289-295; Kusdiana, D. and Saka, S., “Kinetics of transesterification in rapeseed oil to biodiesel fuel as treated in supercritical methanol,” Fuel 80 (2001), 693-698; Saka, S., and Kusdiana, D., “Biodiesel fuel from rapeseed oil as prepared in supercritical methanol,” Fuel 80 (2001), 225-231). The reaction in supercritical methanol reduces the reaction time from 2 hrs to 5 minutes. In addition, the absence of the base catalyst NaOH greatly simplifies the downstream purification, reduces raw material cost, and eliminates the problem with soaps from free fatty acids. Rather than being a problem, the free fatty acids become valuable feedstocks that are converted to biodiesel in the supercritical methanol as follows.

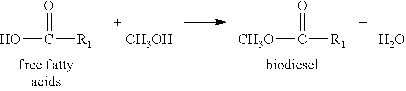

Non-limiting exemplary reaction conditions for both the base-catalyzed and supercritical methanol methods are shown in Table 1 below. As will be apparent to one of ordinary skill in the art, other effective reaction conditions can be applied with routine experimentation to convert the triacylglycerides in fish oil to biodiesel by either one of these methods.

TABLE 1
Comparison between base-catalyzed and supercritical processing

| | Traditional Method | SC Methanol |
|---|---|---|
| Reaction time | 2 hrs | <5 min |
| Conditions | Atmospheric, 60° C. | 1,000 psig, 350° C. |
| Catalyst | NaOH | None |
| FFA product | Soap | Biodiesel |
| Acceptable Water (%) | <1% | No limit |

In another embodiment, triacylglycerides are reduced with hydrogen to produce paraffins, propane, carbon dioxide and water, a product generally known as green diesel. The paraffins can either be isomerized to produce diesel or blended directly with diesel. The primary advantages of hydrogenation over conventional base-catalyzed transesterification are two-fold. First, the hydrogenation process is thermochemical and therefore much more robust to feed impurities as compared to biochemical processes, i.e., hydrogenation is relatively insensitive to free fatty acids and water. Free fatty acids are readily converted to paraffins, and water simply reduces the overall thermal efficiency of the process but does not significantly alter the chemistry. Second, the paraffin product is a pure hydrocarbon, and therefore indistinguishable from petroleum-based hydrocarbons. Unlike biodiesel which has a 15% lower energy content and can freeze in cold weather, green diesel has similar energy content and flow characteristics (e.g., viscosity) to petroleum-based diesel. In some embodiments, the methods provided herein encompass the steps of hydrogenation and isomerization, which are well known in the art to produce liquid fuels, such as jet-fuel, diesel, kerosene, gasoline, JP-1, JP-4, JP-5, JP-6, JP-7, JP-8, and JPTS.

In yet another embodiment, residual fish biomass, such as fishmeal, that remains after the extraction of lipids are used as a feedstock to produce biofuel. Residual fish biomass can be upgraded to bio-oil liquids, a multi-component mixture through fast pyrolysis (for an overview see, e.g., S. Czernik and A.V. Bridgwater, “Overview of Applications of Biomass Fast Pyrolysis Oil,” Energy & Fuels 2004, 18, pp. 590-598; A.V. Bridgwater, “Biomass Fast Pyrolysis,” Thermal Science 2004, 8(8), pp. 21-29); Oasmaa and S. Czernik, “Fuel Oil Quality of Biomass Pyrolysis Oils—State of the Art for End Users,” Energy & Fuels, 1999, 13, 914-921; D. Chiaramonti, A. Oasmaa, and Y. Solantausta, “Power Generation Using Fast Pyrolysis Liquids from Biomass, Renewable and Sustainable Energy Reviews, August 2007, 11(6), pp. 1056-1086). According to certain embodiments provided herein, residual fish biomass is rapidly heated to a temperature of about 500° C., and thermally decomposed to 70-80% liquids and 20-30% char and gases. The liquids, pyrolysis oils, can be upgraded by hydroprocessing to make products, such as naphtha and olefins. Those skilled in the art will know many other suitable reaction conditions, or will be able to ascertain the same by use of routine experimentation.

In yet another embodiment, residual fish biomass can be subjected to gasification which partially oxidizes the biomass in air or oxygen to form a mixture of carbon monoxide and hydrogen or syngas. The syngas can be used for a variety of purposes, such as but not limited to, generation of electricity or heat by burning, Fischer-Tropsch synthesis, and manufacture of organic compounds. For an overview of syngas, see, e.g., Spath, P. L., and Dayton, D.C., “Preliminary Screening—Technical and Ecnomic Assessment of Synthesis Gas to Fuels and Chemicals with Emphasis on the Potential for Biomass-derived Syngas.” NREL/TP-510-34929, December 2003.

In yet another embodiment, residual fish biomass can be subjected to fermentation to convert carbohydrates to ethanol which can be separated using standard techniques. Numerous fungal and bacterial fermentation technologies are known in the art and can be used in accordance with certain embodiments provided herein. For an overview of fermentation, see, e.g., Edgard Gnansounou and Arnaud Dauriat, “Ethanol fuel from biomass: A Review,” Journal of Scientific and Industrial Research, Vol. 64, November 2005, pp 809-821.

In certain embodiments, the processing step involves heating the fishes to greater than about 70° C., 80° C., 90° C. or 100° C., typically by a steam cooker, which coagulates the protein, ruptures the fat deposits and liberates lipids and oil and physico-chemically bound water, and; grinding, pureeing and/or pressing the fish by a continuous press with rotating helical screws. The fishes can be subjected to gentle pressure cooking and pressing which use significantly less energy than that required to obtain lipids from algae. The coagulate may alternatively be centrifuged. This step removes a large fraction of the liquids (press liquor) from the mass, which comprises an oily phase and an aqueous fraction (stickwater). The separation of press liquor can be carried out by centrifugation after the liquor has been heated to 90° C. to 95° C. Separation of stickwater from oil can be carried out in vertical disc centrifuges. In some embodiments, the lipids in the oily phase (fish oil) may be polished by treating with hot water, which extracts impurities from the lipids to form biofuel. To obtain fishmeal, the separated water is evaporated to form a concentrate (fish solubles), which is combined with the solid residues, and then dried to solid form (presscake). The dried material may be ground to a desired particle size. The fishmeal typically comprises mostly proteins (up to 70%), ash, salt, carbohydrates, and oil (about 5-10%). The fishmeal can be used as animal feed and/or as an alternative energy feedstock.

In another embodiment, the fishmeal is subjected to a hydrothermal process that extracts residual lipids, both neutral and polar. A large proportion of polar lipids, such as phospholipids, remain with the fishmeal and lost as biofuel feedstock. Conversion of such polar lipids into fatty acids can boost the overall yield of biofuel from fish. The hydrothermal process provided herein generally comprises treating fishmeal with near-critical or supercritical water under conditions that can extract polar lipids from the fishmeal and/or hydrolyze polar lipids resulting in fatty acids. The fishmeal need not be dried as the moisture in the fishmeal can be used in the process. The process comprises applying pressure to the fish to a predefined pressure and heating the fishmeal to a predefined temperature, wherein lipids in the fishmeal are extracted and/or hydrolyzed to form fatty acids. The fishmeal can be held at one or more of the preselected temperature(s) and preselected pressure(s) for an amount of time that facilitates, and preferably maximizes, hydrolysis and/or extraction of various types of lipids. The term “subcritical” or “near-critical water” refers to water that is pressurized above atmospheric pressure at a temperature between the boiling temperature (100° C. at 1 atm) and critical temperature (374° C.) of water. The term “supercritical water” refers to water above its critical pressure (218 atm) at a temperature above the critical temperature (374° C.). In some embodiments, the predefined pressure is between 5 atm and 500 atm. In some embodiments, the predefined temperature is between 100° C. and 500° C. or between 325° C. and 425° C. The reaction time can range between 5 seconds and 60 minutes. For example, a fishmeal can be exposed to a process condition comprising a temperature of about 300° C. at about 80 atm for about 10 minutes. The selection of an appropriate set of process conditions, i.e., combinations of temperature, pressure, and process time can be determined by assaying the quantity and quality of lipids and free fatty acids, e.g., neutral lipids, phospholipids and free fatty acids, that are produced. The process further comprises separating the treated fishmeal into an organic phase which includes the lipids and/or fatty acids, an aqueous phase, and a solid phase; and collecting the organic phase as biofuel or feedstock.

In some embodiments, the systems provided herein can comprise, independently and optionally, means for gathering fishes from which lipids are extracted (e.g., nets), means for conveying the gathered fishes from the fish enclosure or a holding enclosure to the fish processing facility (e.g., pipes, conveyors, bins, trucks), means for cutting large pieces of fish into small pieces before cooking and pressing (e.g., chopper, hogger), means for heating the fishes to about 70° C., 80° C., 90° C. or 100° C. (e.g., steam cooker); means for grinding, pureeing, and/or pressing the fishes to obtain lipids (e.g., single screw press, twin screw press, with capacity of about 1-20 tons per hour); means for separating lipids from the coagulate (e.g., decanters and/or centrifuges); means for separating the oily phase from the aqueous fraction (e.g., decanters and/or centrifuges); and means for polishing the lipids (e.g., reactor for transesterification or hydrogenation). Many commercially available systems for producing fishmeal can be adapted for use in certain embodiments, including stationary and mobile systems that are mounted on a container frame or a flat rack. The fish oil or a composition comprising fish lipids, can be collected and used as a biofuel, or upgraded to biodiesel or other forms of energy feedstock. For example, biodiesel can be produced by transesterification of the fish lipids, and green diesel by hydrogenation, using technology well known to those of skill in the art.

In certain embodiments, the extracted fish lipids are not limited to use as biofuels. In one embodiment, the extracted fish lipids can be used to obtain Omega-3 fatty acids, such as eicosapentaenoic acid (EPA) and/or docosahexaenoic acid (DHA) and/or derivatives thereof including, but not limited to esters, glycerides, phospholipids, sterols, and/or mixtures thereof. In one embodiment, the extracted fish lipids contain substantially purified EPA and/or DHA ranging from 1 to 50%, depending on the fish species, age, location, and a host of ecological and environmental factors. If higher EPA and/or DHA concentrations are desired, several established methods could be employed, including chromatography, fractional or molecular distillation, enzymatic splitting, low-temperature crystallization, supercritical fluid extraction, ionic liquid extraction/purification, or urea complexation. These methods can further concentrate the EPA and/or DHA to nearly pure EPA and/or DHA.

In certain embodiments, EPA- and/or DHA-containing lipids may be separated and concentrated by short-path distillation, or molecular distillation. The lipids are first transesterified, either acid- or base-catalyzed, with ethanol to produce a mixture of fatty acid ethyl esters (FAEE). The FAEE are then fractionated in the short-path distillation to remove the short chain FAEE, C-14 to C-18. The concentrate of FAEE from C-20 to C-22 is where the EPA and/or DHA can be found. A second distillation of the concentrate can result in a final Omega-3 content of up to 70%. The concentration of the EPA and/or DHA in the final product will depend on the initial lipid profile of the fish oil. The FAEE can be used as a consumer product at this stage (fish oil capsules). In some countries, the FAEE are required to be reconverted to triglycerides through a glycerolysis reaction before they can be sold as a consumer product. In order to obtain pure EPA and/or DHA, an additional purification step is required using chromatography, enzymatic transesterification, ammonia complexation, or supercritical fluid extraction.

In certain embodiments, the systems and methods provide an EPA and/or DHA feedstock or an EPA and/or DHA comprising lipids, hydrocarbons, or both, derived from fish that harvested algae according to the methods provided herein. Lipids of the present embodiments can be subdivided according to polarity: neutral lipids and polar lipids. The major neutral lipids are triglycerides, and free saturated and unsaturated fatty acids. The major polar lipids are acyl lipids, such as glycolipids and phospholipids. The hydrocarbons obtained by the systems and methods provided herein include, but are not limited to, isoprenoids, or pigments such as chlorophyll, carotenoids (e.g., carotene, lycopene, lutein), astaxanthin, melanin, anthocyanins, porphyrins, tetraterpenoids, and betalains. A composition comprising lipids and hydrocarbons of the present embodiments can be described and distinguished by the types and relative amounts of key fatty acids and/or hydrocarbons present in the composition.

Fatty acids are identified herein by a first number that indicates the number of carbon atoms, and a second number that is the number of double bonds, with the option of indicating the position of the first double bond or the double bonds in parenthesis. The carboxylic group is carbon atom 1 and the position of the double bond is specified by the lower numbered carbon atom. For example, EPA is identified as 20:5 (n-3), which is all-cis-5,8,11,14,17-eicosapentaenoic acid, and DHA is identified as 22:6 (n-3), which is all-cis-4,7,10,13,16,19-docosahexaenoic acid, or DHA. The n-3 designates the location of the double bond, counting from the end carbon (highest number).

In certain embodiments, EPA and/or DHA in the predominant form of triglyceride esters can be converted to lower alkyl esters, such as methyl, ethyl, or propyl esters, by known methods and used in an esterification with a sterol to form esters, which can be further purified for use as nutritional supplement. Transesterification, in general, is well known in the art. See, e.g., W. W. Christie, “Preparation of Ester Derivatives of Fatty Acids for Chromatographic Analysis,” Advances in Lipid Methodology—Volume Two, Ch. 2, pp. 70-82 (W. W. Christie, ed., The Oily Press, Dundee, United Kingdom, 1993).

In certain embodiments, to obtain a refined product with higher concentrations of EPA and/or DHA, certain lipases can be used to selectively transesterify the ester moieties of EPA and/or DHA in fish oil triglycerides, under substantially anhydrous reaction conditions, as described in U.S. Pat. No. 5,945,318.

In certain embodiments, one or more edible additives can be included for consumption with the nutritional supplement of containing EPA and/or DHA. In one embodiment, additives can include one or more antioxidants, such as, vitamin C, vitamin E or rosemary extract. In one embodiment, additives can include one or more suitable dispersant, such as, lecithin, an alkyl polyglycoside, polysorbate 80 or sodium lauryl sulfate. In one embodiment, additives can include a suitable antimicrobial such as, for example, sodium sulfite or sodium benzoate. In one embodiment, additives can include one or more suitable solubilizing agent, such as, a vegetable oil such as sunflower oil, coconut oil, and the like, or mono-, di- or tri-glycerides.

In certain embodiments, additives can include, but are not limited to, vitamins such as vitamin A (retinol, retinyl palmitate or retinol acetate), vitamin B1 (thiamin, thiamin hydrochloride or thiamin mononitrate), vitamin B2 (riboflavin), vitamin B3 (niacin, nicotinic acid or niacinamide), vitamin B5 (pantothenic acid, calcium pantothenate, d-panthenol or d-calcium pantothenate), vitamin B6 (pyridoxine, pyridoxal, pyridoxamine or pyridoxine hydrochloride), vitamin B12 (cobalamin or cyanocobalamin), folic acid, folate, folacin, vitamin H (biotin), vitamin C (ascorbic acid, sodium ascorbate, calcium ascorbate or ascorbyl palmitate), vitamin D (cholecalciferol, calciferol or ergocalciferol), vitamin E (d-alpha-tocopherol, or d-alpha tocopheryl acetate) or vitamin K (phylloquinone or phytonadione).

In certain embodiments, additives can include, but are not limited to, minerals such as boron (sodium tetraborate decahydrate), calcium (calcium carbonate, calcium caseinate, calcium citrate, calcium gluconate, calcium lactate, calcium phosphate, dibasic calcium phosphate or tribasic calcium phosphate), chromium (GTF chromium from yeast, chromium acetate, chromium chloride, chromium trichloride and chromium picolinate) copper (copper gluconate or copper sulfate), fluorine (fluoride and calcium fluoride), iodine (potassium iodide), iron (ferrous fumarate, ferrous gluconate gluconate, magnesium hydroxide or magnesium oxide), manganese (manganese gluconate and manganese sulfate), molybdenum (sodium molybdate), phosphorus (dibasic calcium phosphate, sodium phosphate), potassium (potassium aspartate, potassium citrate, potassium chloride or potassium gluconate), selenium (sodium selenite or selenium from yeast), silicon (sodium metasilicate), sodium (sodium chloride), strontium, vanadium (vanadium surface) and zinc (zinc acetate, zinc citrate, zinc gluconate or zinc sulfate).

In certain embodiments, additives can include, but are not limited to, amino acids, peptides, and related molecules such as alanine, arginine, asparagine, aspartic acid, carnitine, citrulline, cysteine, cystine, dimethylglycine, gamma-aminobutyric acid, glutamic acid, glutamine, glutathione, glycine, histidine, isoleucine, leucine, lysine, methionine, ornithine, phenylalanine, proline, serine, taurine, threonine, tryptophan, tyrosine and valine.

In certain embodiments, additives can include, but are not limited to, animal extracts such as cod liver oil, marine lipids, shark cartilage, oyster shell, bee pollen and d-glucosamine sulfate. In certain embodiments, additives can include, but are not limited to, unsaturated free fatty acids such as .gamma.-linoleic, arachidonic and .alpha.-linolenic acid, which may be in an ester (e.g., ethyl ester or triglyceride) form.

In certain embodiments, additives can include, but are not limited to, herbs and plant extracts such as kelp, pectin, Spirulina, fiber, lecithin, wheat germ oil, safflower seed oil, flax seed, evening primrose, borage oil, blackcurrant, pumpkin seed oil, grape extract, grape seed extract, bark extract, pine bark extract, French maritime pine bark extract, muira puama extract, fennel seed extract, dong quai extract, chaste tree berry extract, alfalfa, saw palmetto berry extract, green tea extracts, angelica, catnip, cayenne, comfrey, garlic, ginger, ginseng, goldenseal, juniper berries, licorice, olive oil, parsley, peppermint, rosemary extract, valerian, white willow, yellow dock and yerba mate.

In certain embodiments, additives can include, but are not limited to, enzymes such as amylase, protease, lipase and papain as well as miscellaneous substances such as menaquinone, choline (choline bitartrate), inositol, carotenoids (beta-carotene, alpha-carotene, zeaxanthin, cryptoxanthin or lutein), para-aminobenzoic acid, betaine HCl, free omega-3 fatty acids and their esters, ihiotic acid (alpha-lipoic acid), 1,2-dithiolane-3-pentanoic acid, 1,2-dithiolane-3-valeric acid, alkyl polyglycosides, polysorbate 80, sodium lauryl sulfate, flavanoids, flavanones, flavones, flavonols, isoflavones, proanthocyanidins, oligomeric proanthocyanidins, vitamin A aldehyde, a mixture of the components of vitamin A₂, the D Vitamins (D₁, D₂, D₃ and D₄) which can be treated as a mixture, ascorbyl palmitate and vitamin K₂.

In certain embodiments, fishmeal can be produced from treating fish bodies with a protease acting at a relatively low temperature. In certain embodiments, proteases that can be used include proteinases such as acrosin, urokinase, uropepsin, elastase, enteropeptidase, cathepsin, kallikrein, kininase 2, chymotrypsin, chymopapain, collagenase, streptokinase, subtilisin, thermolysin, trypsin, thrombin, papain, pancreatopeptidase and rennin; peptidases such as aminopeptidases, for example, arginine aminopeptidase, oxytocinase and leucine aminopeptidase; angiotensinase, angiotensin converting enzyme, insulinase, carboxypeptidase, for example, arginine carboxypeptidase, kininase 1 and thyroid peptidase, dipeptidases, for example, carnosinase and prolinase and pronases; as well as other proteases, denatured products thereof and compositions thereof.

Technical and scientific terms used herein have the meanings commonly understood by one of ordinary skill in the art to which the present systems and methods pertain, unless otherwise defined. Reference is made herein to various methodologies known to those of skill in the art. Publications and other materials setting forth such known methodologies to which reference is made are incorporated herein by reference in their entireties as though set forth in full. The practice of certain embodiments provided herein will employ, unless otherwise indicated, techniques of chemistry, biology, the aquaculture industry and the algae industry, which are within the skill of the art. Such techniques are explained fully in the literature, e.g., Aquaculture Engineering, Odd-Ivar Lekang, 2007, Blackwell Publishing Ltd.; Handbook of Microalgal Culture, edited by Amos Richmond, 2004, Blackwell Science; Microalgae Biotechnology and Microbiology, E.W. Becker, 1994, Cambridge University Press; Limnology: Lake and River Ecosystems, Robert G. Wetzel, 2001, Academic Press, each of which are incorporated by reference in their entireties.

All references cited herein are incorporated herein by reference in their entirety and for all purposes to the same extent as if each individual publication or patent or patent application was specifically and individually indicated to be incorporated by reference in its entirety for all purposes.

Many modifications and variations of the embodiments provided herein can be made without departing from its spirit and scope, as will be apparent to those skilled in the art. The specific embodiments described herein are offered by way of example only, and the embodiments are to be limited only by the terms of the appended claims along with the full scope of equivalents to which such claims are entitled.

What is claimed:

1. A method for producing a biofuel from microalgae, said method comprising:
(i) providing one or more populations of zooplankton and one or more populations of planktivorous fishes; wherein zooplankton in at least one of said populations of zooplankton feed on microalgae, thereby harvesting the microalgae;
(ii) gathering the planktivorous fishes in said one or more populations of planktivorous fishes that feed on (a) said one or more populations of zooplankton, and/or (b) said one or more populations of zooplankton and the microalgae;
(iii) extracting lipids from (c) the zooplankton, and/or (d) the planktivorous fishes, that have been gathered; and
(iv) processing the lipids to form said biofuel.

2. The method of claim 1, wherein at least one of said one or more populations of zooplankton comprise zooplankton that are about 200 μm to about 2 mm in dimensions.

3. The method of claim 1, wherein at least one of said one or more populations of zooplankton comprise zooplankton that are about ten times the dimensions of a dominant species of microalgae that is being harvested.

4. The method of claim 1, wherein at least one of said one or more populations of zooplankton comprise copepods and/or cladocerans.

5. The method of claim 1, wherein at least one of said one or more populations of zooplankton comprise pelagic tunicates.

6. The method of claim 1, wherein at least one of said one or more populations of zooplankton comprise larvae of fishes and/or larvae of shellfishes.

7. A method for producing a biofuel from microalgae, said method comprising:
(i) harvesting microalgae by one or more populations of zooplankton that feed on the microalgae;
(ii) extracting lipids from said one or more populations of zooplankton; and
(iii) processing the lipids to form said biofuel.

8. The method of claim 7, wherein at least one of said one or more populations of zooplankton comprise zooplankton that are about 200 μm to about 2 mm in dimensions.

9. The method of claim 7, wherein at least one of said one or more populations of zooplankton comprise zooplankton that are about ten times the dimensions of a dominant species of microalgae that is being harvested.

10. The method of claim 7, wherein at least one of said one or more populations of zooplankton comprise copepods and/or cladocerans.

11. The method of claim 7, wherein at least one of said one or more populations of zooplankton comprise pelagic tunicates.

12. The method of claim 7, wherein at least one of said one or more populations of zooplankton comprise larvae of fishes and/or larvae of shellfishes.

13. A method for producing an eicosahexaenoic acid (EPA) and/or docosahexaenoic acid (DHA) and/or derivatives thereof from microalgae, said method comprising:
(i) providing one or more populations of zooplankton and one or more populations of planktivorous fishes; wherein zooplankton in at least one of said populations of zooplankton feed on microalgae, thereby harvesting the microalgae;
(ii) gathering the planktivorous fishes in said one or more populations of planktivorous fishes that feed on (a) said one or more populations of zooplankton, and/or (b) said one or more populations of zooplankton and the microalgae;
(iii) extracting lipids from (c) the zooplankton, and/or (d) the planktivorous fishes, that have been gathered; and
(iv) processing the lipids to form EPA and/or DHA and/or derivatives thereof.

14. The method of claim 1, wherein at least one of said one or more populations of zooplankton comprise zooplankton that are about 200 μm to about 2 mm in dimensions.

15. The method of claim 1, wherein at least one of said one or more populations of zooplankton comprise zooplankton that are about ten times the dimensions of a dominant species of microalgae that is being harvested.

16. The method of claim 1, wherein at least one of said one or more populations of zooplankton comprise copepods and/or cladocerans.

17. The method of claim 1, wherein at least one of said one or more populations of zooplankton comprise pelagic tunicates.

18. The method of claim 1, wherein at least one of said one or more populations of zooplankton comprise larvae of fishes and/or larvae of shellfishes.

19. A method for producing an eicosahexaenoic acid (EPA) and/or docosahexaenoic acid (DHA) and/or derivatives thereof from microalgae, said method comprising:
(i) harvesting microalgae by one or more populations of zooplankton that feed on the microalgae;
(ii) extracting lipids from said one or more populations of zooplankton; and
(iii) processing the lipids to form EPA and/or DHA and/or derivatives thereof.

20. The method of claim 7, wherein at least one of said one or more populations of zooplankton comprise zooplankton that are about 200 μm to about 2 mm in dimensions.

21. The method of claim 7, wherein at least one of said one or more populations of zooplankton comprise zooplankton that are about ten times the dimensions of a dominant species of microalgae that is being harvested.

22. The method of claim 7, wherein at least one of said one or more populations of zooplankton comprise copepods and/or cladocerans.

23. The method of claim 7, wherein at least one of said one or more populations of zooplankton comprise pelagic tunicates.

24. The method of claim 7, wherein at least one of said one or more populations of zooplankton comprise larvae of fishes and/or larvae of shellfishes.

25. A system for producing a biofuel comprising an enclosure comprising microalgae, means for feeding microalgae to one or more populations of zooplankton, means for extracting lipids from zooplankton, and means for processing the lipids into said biofuel.

26. A system for producing a biofuel comprising an enclosure comprising microalgae, one or more populations of zooplankton and one or more populations of fishes, means for feeding microalgae to said one or more populations of zooplankton, means for gathering zooplankton and/or fishes, means for extracting lipids from fishes, and means for processing the lipids into said biofuel.

27. A system for producing an EPA and/or DHA and/or derivatives thereof comprising an enclosure comprising microalgae, means for feeding microalgae to one or more populations of zooplankton, means for extracting lipids from zooplankton, and means for processing the lipids into said EPA and/or DHA and/or derivatives thereof.

28. A system for producing an EPA and/or DHA and/or derivatives thereof comprising an enclosure comprising microalgae, one or more populations of zooplankton and one or more populations of fishes, means for feeding microalgae to said one or more populations of zooplankton, means for gathering zooplankton and/or fishes, means for extracting lipids from fishes, and means for processing the lipids into said EPA and/or DHA and/or derivatives thereof.

29. A composition comprising lipids, said composition being prepared by extracting lipids from zooplankton that had been fed with cultured microalgae.
